(19) United States
(12) Patent Application Publication
(10) Pub. No.: US 2011/0256239 A1
(43) Pub. Date: Oct. 20, 2011

(54) INGESTIBLE HAIR AND FINGER NAIL GROWTH PROMOTER AND METHOD

(76) Inventor: Lawrence J. Shapiro, Delray Beach, FL (US)

(21) Appl. No.: 13/087,372

(22) Filed: Apr. 14, 2011

Related U.S. Application Data
(60) Provisional application No. 61/324,607, filed on Apr. 15, 2010.

(51) Int. Cl. A61K 38/16; A61P 17/14; A61P 17/00; A61K 36/704

(52) U.S. Cl. 424/641; 514/20.7

(57) ABSTRACT

A composition and method for promoting hair growth comprising oral administration of a whey protein based formulation containing particular percentages of vitamins and supplements from between 2 weeks to 6 months.

This application claims benefit to U.S. Provisional Application No. 61/324,607 filed Apr. 15, 2010, the disclosure of which is incorporated herein by reference in its entirety.

The present invention relates to a composition for promoting hair and finger nail growth and preventing hair loss. The present invention also relates to a method for making and using said composition.

### BACKGROUND OF THE INVENTION

Numerous devices, medical procedures and pharmaceutical applications have been created for the treatment of hair loss. For example, U.S. Pat. No. 7,201,764 to Pearl discloses a hand-held laser device designed to stimulate hair growth. The device provides distributed laser light to the scalp while simultaneously parting the individual's hair to ensure that the laser light contacts the individual's scalp.

Others have also created dietary supplements and vitamins to provide the body with the building blocks necessary to grow healthy hair and nails. However, many of these products have incorporated ingredients that have undesired side effects, or include ingredients that have been known to cause hair loss or adversely affect thyroid function, such as products that incorporate soy proteins. Additionally, many hair products incorporate whey protein isolates that have been shown to have adverse effects on hair growth. Many powdered protein supplements include whey protein isolates that can cause a hormonal imbalance leading to hair loss. Many nutritional supplements intentionally incorporate whey protein isolates because such isolates are considered the most pure and concentrated form of whey protein available with very little fat and lactose content. This type of protein is often desired because of beneficial affects related to the repair of skeletal muscles resulting from strenuous and extreme athletic activity. However, the isolate form can also cause unintended hair loss due to the build-up of testosterone in the body. Whey protein isolates, which have a higher amino acid and protein content, also produce higher testosterone levels, causing higher levels of DHT, which is the breakdown of the testosterone into dihydrotestosterone. The presence of dihydrotestosterone has been associated with weaker and smaller hair follicle growth, and eventually, with hair loss.

There is a need for a dietary supplement that promotes hair and finger nail growth that does not use whey protein isolates or soy-based ingredients.

There is a need for an ingestible composition that promotes hair and finger nail growth that does not cause undesired side effects.

### OBJECTS OF THE INVENTION

The following section of the written description describes some of the objects of the present invention, but the section is not exhaustive of all of invention's objects.

It is an object of the present invention to provide an ingestible composition of matter that can consumed by a person to help that person's hair grow and look thicker, fuller, stronger and healthier, and to make nails grow healthier and stronger.

It is a further object of the present invention to provide a hair-growth composition that includes essential amino acids, vitamins and minerals that are the building blocks of the hair and nails.

It is yet another objection of the present invention to provide an ingestible hair and finger nail growth enhancing product that comprises an enhanced whey protein.

It is a further object of the present invention to provide an ingestible hair and finger nail growth enhancing product that incorporates a whey protein concentrate that prevents hair loss due to genetics, menopause, post-pregnancy, weight loss products, workout products, telogen effluvium, chemotherapy, and hair dormancy and shock loss after hair transplantation.

### SUMMARY OF THE INVENTION

The composition of one embodiment of the present invention comprises an enhanced whey protein concentrate that is combined with biotin.

The composition of another embodiment of the present invention comprises an enhanced whey protein concentrate that is combined with biotin, vitamins, minerals, amino acids, Chinese herbs and seeds in proportions that are designed specifically to aid the growth of hair and nails.

The composition of another embodiment of the present invention comprises an enhanced whey protein concentrate that is all natural, hormone free and fortified with vitamins, minerals, amino acids, Chinese herbs and seeds designed specifically to aid the growth of hair and nails for individuals prone to hair loss induced by genetics, menopause, post-pregnancy, weight loss products, workout products, telogen effluvium, chemotherapy, and hair dormancy and shock loss after hair transplantation.

### DETAILED DESCRIPTION OF THE PREFERRED EMBODIMENTS

The present invention relates to a composition comprising an enhanced whey protein concentrate that is combined with biotin. Another embodiment of the present invention comprises an enhanced whey protein concentrate that is combined with biotin, vitamins, minerals, amino acids, Chinese herbs and seeds in proportions that are designed specifically to aid the growth of hair and nails. The composition of another embodiment of the present invention comprises an enhanced whey protein concentrate that is all natural, hormone free and fortified with vitamins, minerals, amino acids, Chinese herbs and seeds designed specifically to aid the growth of hair and nails for individuals prone to hair loss induced by genetics, workout products, weight loss products, post-pregnancy and menopause and thus, helping prevent hair loss and dormancy in hair transplants.

It is important to note that the embodiments of the invention described herein are only examples of some of the uses of the teachings described herein. In general, statements made in the specification do not limit any of the various claimed inventions.

The composition of the present invention may be embodied in a several forms, such as a dietary supplement or a dry powder-like composition that may be mixed with a beverage. The composition may be ingested by a person as a supplement to a meal or may be fortified with other vitamins, minerals and proteins such that it may utilized as a snack or a meal replacement. For example, embodiments of the composition may include: 1) a workout supplement; 2) snack/meal replacement for weight loss; 3) aid in preventing post-pregnancy/post-partum hair loss and/or thinning; 4) used to replace products that utilize a whey protein isolate; 5) an aid in preventing hair loss or thinning normally occurring in menopause; 6) used as a healthy alternative for growth hormone free whey; 7) aid in promoting the growth of thicker, stronger, longer and healthier hair; longer and stronger nails; and 8) aid in decreasing the dormancy period after hair transplantation.

One embodiment of the present invention relates to a composition comprising an enhanced whey protein concentrate that is combined with biotin. The whey protein concentrate, unlike the whey protein isolates, provides a protein that enhances hair growth. The whey protein concentrate does not have the adverse side effect produced by the whey protein isolates. Biotin is a water-soluble B-complex vitamin that is necessary for cell growth. Biotin is often recommended for strengthening hair and nails. Hence, this combination provides an excellent ingestible composition that promotes the growth of healthy hair and finger nails.

Another embodiment of the present invention comprises an enhanced whey protein concentrate that is combined with biotin, vitamins, minerals, amino acids, Chinese herbs and seeds in proportions that are designed specifically to aid the growth of hair and nails. The following tables provide a list of ingredients and quantities, plus instructions on how to make a chocolate flavored ingestible composition comprising exemplary embodiments of the present invention:

TABLE 1
INGREDIENTS

| Item # | Description | Formula Weight | |
|---|---|---|---|
| 1 | WHEY PROTEIN | 72.6 | KG |
| 2 | CHOCOLATE FLAVOR | 9.9 | KG |
| 3 | BIOTIN 1% | 825 | GM |
| 4 | POTASSIUM IODIDE | 3.33 | GM |
| 5 | NIACINAMIDE | 132 | GM |
| 6 | FOLC ACID 10% | 13.2 | GM |
| 7 | PABA | 99 | GM |
| 8 | VITAMIN B5 | 303 | GM |
| 9 | STEVIA | 330 | GM |
| 10 | ZINC OXIDE | 561 | GM |
| 11 | XYLITOL | 13.2 | KG |
| 12 | MANGANESE CITRATE | 66 | GM |
| 13 | FOTI | 82.5 | GM |
| 14 | KUDZU ROOT | 66 | GM |
| 15 | PUMPKIN SEED | 33 | GM |
| 16 | HONEYSUCKLE | 16.5 | GM |
| 17 | CHRYSANTHEMUM FLOWER | 16.5 | GM |
| 18 | SILICA | 3.3 | GM |
| 19 | L-METHIONINE | 3.3 | GM |
| 20 | L-CYSTEINE | 99 | GM |
| 21 | VITAMIN B12 (CYANO) | 3 | GRAMS |
| | TOTAL | 98.35 | KG |

TABLE 2
Mixing Instructions
Procedure:

2. Weigh items 1-21 and pour into mixer.
3. Blend items 1-21 in mixer for 20 minutes.

Although one embodiment provides blending for 20 minutes, the blend time is adjusted as necessary such that the final blend exhibits acceptable content uniformity.

In one embodiment, the mixing is carried out as a dry blend in appropriate mixing apparatus.

However, in order to achieve acceptable content uniformity, mixing can also be carried out using granulation techniques including, but not limited to fluid bed granulation, vertical granulation, high shear processing, extrusion and others processing techniques. The composition of another embodiment of the present invention comprises an enhanced whey protein concentrate that is all natural, hormone free and fortified with vitamins, minerals, amino acids, Chinese herbs and seeds designed specifically to aid the growth of hair and nails for individuals prone to hair loss induced by genetics, workout products, weight loss products, post-pregnancy and menopause and thus, helping prevent hair loss and dormancy in hair transplants.

The following Table 3 are additional ingredients that provide the benefits related to the growth of hair and finger nails:

TABLE 3

| Mg per serving | Description |
|---|---|
| 25 mg | FOTI |
| 20 mg | KUDZU ROOT |
| 10 mg | PUMPKIN SEED |
| 5 mg | HONEYSUCKLE |
| 5 mg | CHRYSANTHEMUM FLOWER |
| 1 mg | SILICA |
| 1 mg | L-METHIONINE |
| 30 mg | L-CYSTEINE |

The amount of ingredients included in the composition of the present invention may be modified and still achieve the desired results. For example, the amount of vitamins included in a single serving may vary as follows:

TABLE 4
Vitamin Ranges

| | |
|---|---|
| Niacin | 10-60 mg |
| Folate | 100-600 mcg |
| Vitamin B-12 | 1.5-8 mcg |
| Biotin | 250 mcg-6 mg |
| Pantothenic acid | 2 MG-12 mg |
| Iodine | 25 mcg-200 mcg |
| Zinc | 5 mg-30 mg |
| Maganese | 0.5 MG-4 mg |
| Chlorophyll | 2 mg-20 MG |
| PABA | 7 mg-50 MG |

Similarly, the minerals, herbs and seeds may vary as follows:

TABLE 5

| | |
|---|---|
| FOTI | 1-50 mg |
| KUDZU ROOT | 1-40 mg |
| PUMPKIN SEED | 1-30 mg |
| HONEYSUCKLE | 1-15 mg |
| CHRYSANTHEMUM FLOWER | 1-15 mg |
| SILICA | 1-10 mg |
| L-METHIONINE | 1-30 mg |
| L-CYSTEINE | 1-60 mg |

The present invention has unexpectedly achieved a surprising synergistic result by providing a dosage in which several components are administered below the accepted recommended daily allowance (RDA) yet contribute to the desired therapeutic effect of promoting scalp hair regrowth.

Typically, RDA values are determined based on the body's ability to process components as well as considering non-processory effect including, but not limited to hepatic metabolism, elimination, accumulation and other physiological effects. Often, combinations containing numerous components are administered with components at or exceeding the RDA values. This is because combinations often have an interfering effect on each other and prevent full therapeutic effect even at or over RDA values.

As shown below, a 32 g dose prepared according to the example above in Table 1 demonstrates the desired therapeutic effect, while several components are below RDA

TABLE 6

| | RDA | | Amount per 32 g dose | |
|---|---|---|---|---|
| Biotin | 20-30 | μg/day | 2.68 | μg |
| Folic Acid | 300-400 | μg/day | 4.22 | μg |
| PABA | 50 | mg/day | 32.2 | mg |
| *L-Methionine | | | 1.07 | mg |
| *L-Cysteine | | | 32.21 | mg |

*L-Methionine and L-Cysteine have a combined RDA of 500-700 mg/day.

Efficacy of the present invention is established by clinical testing.

The subjects were adults with thinning hair, of various ages and both genders. They were selected at random. They were all healthy with no serious underlying medical conditions. A full medical history of each subject was taken.

The majority of the subjects were men experiencing androgenetic alopecia (male pattern balding). Androgenetic alopecia has strong genetic causes, but its onset and severity may be influenced by nutrition. In men with male pattern hair loss, the follicles on the top and front of the scalp are genetically pre-programmed to become progressively sensitive to the hormone Dihydrotestosterone (DHT). When these hair follicles are exposed to excessive DHT, they no longer go through the normal growth cycle. The duration of the anagen (or growth) phase becomes progressively shorter, and this results in the hair from an affected follicle becoming thin and short. Ultimately, the follicle will enter the telogen phase but will not re-awaken, resulting in hair loss. Androgenetic alopecia is progressive in nature, and hair loss continues until a “wreath” of hair on the sides and back of the scalp remains.

Six of the females in the study had post-pregnancy hair loss. Nine of the women were menopausal with hair loss, and seven of these nine women had underlying thyroid problems that had been corrected prior to the start of the study.

All the subjects had thinning hair, but were not completely bald. Even if they had hair in one area that was thin and an area that was bald, they could participate in the study. Subjects were not compensated and were given a supply of Help Hair Shake.

The subjects took a composition prepared according to Table 1 (the test composition) daily for a minimum of 4 weeks. Most of the subjects ingested between 1 and 2 scoops (32 g-64 g) of the test composition in water or a beverage each day.

After a minimum of 4 weeks of dosing the test composition, the subjects completed a survey about the quality of their hair and nails. The survey also asked about their prior use of whey protein isolate. If the subjects had taken whey protein isolates for at least 4 weeks in the past, they indicated how it affected their hair.

For the evaluation of the subjects' hair after taking the test composition, the survey utilized a scale of 1-10, in which 1 meant “no change “, 6 meant “some change” and 10 meant “strong change noticed.” The survey asked whether their hair looked healthier, shinier, less hair loss, less breakage, stronger, longer, grew faster, bouncier, fuller and thicker.

To evaluate the nails, the survey utilized a scale of 1-10, in which 1 meant “no change”, 6 meant “some change” and 10 meant “strong change noticed.” The survey asked whether the subjects' nails were stronger, longer, healthier, less breakage, and faster growth.

The survey asked if the subjects had taken protein shakes containing whey protein isolate for at least 4 consecutive weeks in the past. If they had, a scale of 1-10 was utilized, in which 1 meant “strongly applicable—strong effect noticed”, 6 meant “moderate effect” and 10 meant “no difference noticed.” Additionally, the survey asked whether subjects' whey protein isolate shakes had been associated with hair loss, breakage, dullness, weaker hair, less bounce, thinner and slower growth.

Additionally, the subjects were asked about their reported lost weight, built muscle, more energy and reduced appetite on a scale of 1-10, in which 1 meant “no change “, 6 meant “some change” and 10 meant “strong change noticed.”

Test Results

Nearly all subjects (94%) noticed some improvement in their hair's appearance after taking the test composition for at least 4 weeks, with a score of 6 or higher in each category: healthy, shiny, less hair loss, less hair breakage, stronger, longer, grew faster, bouncier, fuller and thicker (Table 7). The mean score for all categories ranged from 7.5 to 7.8, with a mean overall score of 7.7. Overall, 62% of participants noticed significant improvement, giving a score of 8 or higher.

Three-quarters of the subjects (n=74) had taken supplements containing whey protein isolate in the past for at least 4 weeks (Table 9). Of these subjects, 95% noticed moderate or worse hair damage from taking whey protein isolates, including hair loss, breakage, dullness, weaker hair, less bounce, thinner and slower growth. Significant damage was reported by 30% of the subjects: hair loss (11%), breakage (12%), dullness (14%), slower growth (18%), less bounce (19%), weaker hair (20%), and thinner (22%).

Nail changes were also reported in the survey. After taking Help Hair Shake, an embodiment of the present invention, 93% of the subjects noticed some improvement in their nails' strength, length, health, less breakage and faster growth (Table 8). The mean score for all categories ranged from 7.6 to 7.7, with a mean overall score of 7.7. Overall, 66% of participants noticed significant nail improvement, giving a score of 8 or higher.

In terms of muscle building and weight loss, approximately 84% of the subjects reported weight loss, 85% reported built muscle, 85% noticed increased energy and 83% indicated a reduced appetite after taking Help Hair Shake for at least 4 weeks (Table 4). The mean score for all categories ranged from 7.3 to 7.5. Significant improvement in lost weight was noticed by 51% of the subjects; “built muscle” was 47%; “more energy” was 55%; and “reduced appetite” was 53%.

The taste of the product was reportedly “good” or better by 83% of subjects, and 53% indicated an “excellent” taste (Table 10).

Subgroups

Six female subjects who had post pregnancy hair loss indicated improvement from the test composition.

Nine female subjects were menopausal and had hair loss; of these, seven had underlying thyroid problems that were corrected prior to the start of the study. They all indicated improvement with the test composition.

(1) Tables and Graphs

TABLE 7
Test Composition Effect on Hair

| | | | % of Subjects | Total: % of |
|---|---|---|---|---|
| | | % of Subjects | Noticing | Subjects |
| | | Noticing Some | Significant | Noticing |
| | | Improvement | Improvement | Improvement |
| | Mean | (Score of | (Score of 8 or | (Score of 6 or |
| n = 100 | Score | 6 or 7) | Higher) | Higher) |
| Healthy | 7.8 | 37% | 55% | 92% |
| Shiny | 7.7 | 36% | 53% | 89% |
| Less hair loss | 7.5 | 36% | 49% | 85% |
| Less hair | 7.6 | 36% | 49% | 85% |
| breakage | | | | |
| Stronger | 7.8 | 36% | 55% | 91% |
| Longer | 7.7 | 35% | 56% | 91% |
| Grew faster | 7.7 | 31% | 56% | 87% |
| Bouncier | 7.7 | 39% | 48% | 87% |
| Fuller | 7.7 | 33% | 54% | 87% |
| Thicker | 7.6 | 37% | 51% | 88% |
| Overall Results | 7.7 | 32% | 62% | 94% |

TABLE 8
Test Composition Effect on Nails

| | | % of Subjects | % of Subjects | Total: % of |
|---|---|---|---|---|
| | | Noticing | Noticing | Subjects |
| | | Some | Significant | Noticing |
| | | Improvement | Improvement | Improvement |
| | Mean | (Score of | (Score | (Score of 6 or |
| n = 100 | Score | 6 or 7) | of 8 or Higher) | Higher) |
| Stronger | 7.7 | 29% | 62% | 91% |
| Longer | 7.7 | 25% | 62% | 87% |
| Healthier | 7.7 | 29% | 60% | 89% |
| Less Breakage | 7.6 | 37% | 51% | 88% |
| Faster Growth | 7.7 | 27% | 61% | 88% |
| Overall | 7.7 | 27% | 66% | 93% |
| Results | | | | |

TABLE 9
Whey Protein Isolate Effect on Hair

| | | % of Subjects | |
|---|---|---|---|
| | | Noticing Moderate | % of Subjects Noticing |
| | Mean | Damage (Score of 5 | Significant Damage |
| n = 74 | Score | or Lower) | (Score of 3 or Lower) |
| Hair Loss | 4.9 | 88% | 11% |
| Breakage | 4.8 | 86% | 12% |
| Dullness | 4.8 | 86% | 14% |
| Weaker hair | 4.6 | 93% | 20% |
| Less bounce | 4.7 | 91% | 19% |
| Thinner | 4.6 | 91% | 22% |
| Slower growth | 4.7 | 91% | 18% |
| Overall | 4.7 | 95% | 30% |
| Results | | | |

TABLE 10
Effects of Test Composition on Weight, Muscle, Energy and Appetite

| | | | % of Subjects | Total: % |
|---|---|---|---|---|
| | | | Noticing | of Subjects |
| | | % of Subjects | Significant | Noticing |
| | | Noticing Some | Improvement | Improvement |
| | Mean | Improvement | (Score | (Score |
| n = 99 | Score | (Score of 6 or 7) | of 8 or Higher) | of 6 or Higher) |
| Lost Weight | 7.3 | 33% | 51% | 84% |
| Built Muscle | 7.3 | 37% | 57% | 85% |
| More Energy | 7.5 | 30% | 55% | 85% |
| Reduced | 7.4 | 30% | 53% | 83% |
| Appetite | | | | |

TABLE 11
Taste of Test Composition

| | | % of Subjects | % of Subjects | |
|---|---|---|---|---|
| | | Noting Good | Noting | Total: % of Subjects |
| | | Taste | Excellent Taste | Noting Good or |
| | Mean | (Score of | (Score of 8 | Excellent Taste |
| n = 99 | Score | 6 or 7) | or Higher) | (Score of 6 or Higher) |
| Taste | 7.7 | 30% | 53% | 83% |

(2) Discussion

The test composition was shown to significantly improve perceived hair health, shine, hair loss, breakage, strength, length, growth rate, bounce, fullness and thickness for the majority of the subjects, all of whom had experienced hair loss. Perceived nail quality also improved, with the majority of subjects reporting that their nails were stronger, longer, healthier, had less breakage, and had faster growth.

Contrarily, whey protein isolate supplements are strongly associated with perceived hair loss, breakage, dullness, weaker hair, less bounce, thinner and slower growth.

Nutritional supplementation can have a strong positive or negative impact on perceived and actual hair health. Whey protein isolate is easy to mix with fluids, and it is used in most whey supplements. Whey protein isolate is a highly processed substance and may contain extra growth hormones from the dairy product. This substance often leads to hair damage, and may be linked to premature hair loss in genetically prone individuals.

The whey protein concentrate formulation used in this study, the test composition, is made with whey protein concentrate, which is whey in a more natural state. The dairy product comes from cows that were not injected with rBGH and this may reduce the amount of the growth hormone IFG-1 in the product to natural levels.

The test composition contains additional nutrients that may be associated with improved hair, including biotin, B vitamins, zinc, and an amino acid blend. The formula contains the Chinese herbal remedies Kudzo Root and Fo-ti Root (Ho Shou Wu) which are traditionally used for many ailments including hair problems. Help Hair Shake also includes pumpkin seed, honeysuckle flower and chrysanthemum flower.

In addition to the more natural form of whey and the extra ingredients, the test composition is a completely natural product, with no artificial sweeteners, no xanthan gum, no MSG, no artificial colors, and no artificial flavors. It contains the natural sweeteners stevia and xylitol. Stevia has a negligible effect on blood sugar and is thought to be useful for certain hair conditions.

Additionally, it has been found that the composition and method of the present invention are effective in treating different types of hair loss.

Existing, non-transplanted hair is often damaged from the hair transplant due to trauma from proximate transplanted hair. This damage of existing hair is called shock loss. Often, the blade used in transplant has a damaging effect the existing hair. Furthermore, existing, non-transplanted hair exhibits trauma from the local anesthesia.

Telogen effluvium is a scalp disorder characterized by massive hair loss as a result of early entry of hairs into the telogen phase. Emotional or physiological stressful events may result in an alteration of the normal hair cycle. It may be due to a variety of causes, eating disorders, fever, childbirth, chronic illness, major surgery, anemia, severe emotional disorders, crash diets, hypothyroidism, and drugs.

Physiologic stress is the cause of telogen effluvium.

These inciting factors can be organized into several categories, noted below. Evidence from mouse studies indicates that psychological stress can induce catagen, mainly by effects on neurotransmitters and hormones In humans, however, the role these effects play in hair loss has not yet been determined. While substance P has been extensively studies in human hair follicles in vitro, in vivo studies have not been performed.7 In HIV disease, apoptosis may be related to HIV-1 viral protein R.8. Note the following causes:
Acute illness such as febrile illness, severe infection, major surgery, and severe trauma
Chronic illness such as malignancy, particularly lymphoproliferative malignancy; and any chronic debilitating illness, such as systemic lupus erythematosus, end-stage renal disease, or liver disease. Hormonal changes such as pregnancy and delivery (can affect both mother and child), hypothyroidism, and discontinuation of estrogen-containing medications (see image below)
Telogen effluvium secondary to hypothyroidism.
Changes in diet like crash dieting, anorexia, low protein intake, and chronic iron deficiency; a study by Olsen, et al sought to determine if iron deficiency played a role in female pattern hair loss. Results indicated that iron deficiency is common in women but is not significantly increased in patients with female patterns of hair loss or chronic telogen effluvium when compared with control subjects.
Heavy metals such as selenium, arsenic, and thallium.
Medications, of which the most frequency cited are beta-blockers, anticoagulants, retinoids (including excess vitamin A), propylthiouracil (induces hypothyroidism), carbamazepine, and immunizations.
Allergic contact dermatitis of the scalp.

Telogen effluvium also associated with menopause and hair loss after surgery.

Testing Methods and Results for Telogen Effluvim

A study was conducted in which a growth hormone free enhanced whey protein concentrate (34%) according to the present invention was used to decrease the dormancy period after the hair transplantation. It is suspected that whey protein isolate had caused premature hair loss and that anabolics in general will elevate testosterone and DHT levels causing premature hair loss. The anabolics include but not exclusive ingestible as creatine, andro, testosterone patches, steroids, growth hormone, and fat burners which change the metabolism. Whey protein is derived when milk is separated into casein and fat soluble whey protein. Whey protein consists of lactose, minerals, calcium, phosphate, lacto albumin, lacto globulin which is found in whey protein concentrate. This is further processed by various filtration methods to create Whey protein isolate (80%-100%), which has a higher amino acid and protein content removing the milk sugars and minerals versus the more natural whey protein concentrate (WPC). One study shown below specifically connects branched chains amino acids to increased testosterone production. By using a growth hormone-free whey protein concentrate formula (34%) according to the present invention, a product with a lower amino acid count is used which is less androgenic without the side effect of premature hair loss.

The present invention enhanced the WPC with a variety of amino acids, vitamins and minerals.

When the hair transplant patients were given a preparation of the present invention concurrent and immediately after surgery, with continued daily dosing, within 5 days after surgery, transplanted hair grew in faster than had been previously observed.

After a hair transplant, the transplanted hair follicles enter the dormancy (telogen) phase for 3 to 6 month before they start to grow (anagen phase). Full growth is usually achieved in 10 to 12 months. Until now, nothing has been known to reduce the length of this dormancy phase, nor to eliminate this phase. An all natural approach to reducing or eliminating the telogen phase would be highly valued by hair transplant patients seeking faster results.

The growth hormone-free whey protein concentrate formulation (34%) used in this study and prepared according to the present invention, contains a particular concentration and combination of vitamins, minerals, herbs and amino acids beneficial to hair growth. This study of 115 men and women who took the enhanced whey protein concentrate formulation of the present invention (referred to as EWPC) as directed immediately after hair transplantation. Within 5 days after a hair transplant, all test subjects—100%—had hair starting to grow from the transplanted follicles of 1-2 hairs just 6 weeks (1.5 months) after their hair transplant. At 12 weeks (3 months), all these subjects (100%) had at least ¾″ length of 1-2 hairs in the transplanted area. Combining a hair transplant with the EWPC to decrease the dormancy period is called Accelerated Follicular Restoration (AFR).

The 115 controls, who had a hair transplant without taking the EWPC had the typical results with their hair transplant: little or no significant growth at 6 weeks and little or no significant growth at 12 weeks, with growth starting after that time. Both groups were prescribed finesteride 1 mg daily p.o. 5 days after surgery and topical Rogaine 2% one month after surgery.

EWPC is the first product known to reduce the length of the telogen phase after a hair transplant in some individuals.

As discussed above, after a hair transplant, the transplanted hair follicles enter the dormancy (telogen) phase for 3 to 6 month before they start to grow (anagen phase). Full growth is usually achieved in 10 to 12 months. Until now, nothing has been known to reduce the length of this dormancy phase, nor to eliminate this phase. The results of the study using the present invention show that a special whey protein concentrate formulation can reduce the length of the dormancy phase after a hair transplant.

When hair follicles are transplanted from one part of the scalp to another, the hair is trimmed before being transplanted. Any hairs that remain in the transplanted follicles usually fall out due to shock loss, and the follicle enters the telogen phase. The hair transplant patient must wait at least 3 to 6 months before seeing hairs grow in any of these transplanted follicles. In rare cases, growth happens sooner. The hair transplant takes full effect 10 to 12 months after the surgery.

People seeking hair transplants are looking forward to the benefits of a fuller head of hair and are usually eager to seeing the results sooner than nature provides without the benefits of the composition of the present invention. Waiting up to one year for complete results reduces the time that patients can enjoy the benefits of their transplant. Reducing this waiting period would be highly sought by hair transplant candidates. On the surface, drug research may seem appropriate for reducing the hair transplant dormancy period. So far, no drug has been shown to significantly decrease the dormancy period of a hair transplant. Moreover, drugs run the risk of side effects. Patients worry about the possibility of doing more harm than good. An all natural, proven approach to accelerating the results from a hair transplant would likely have more acceptance and higher compliance than a drug.

Hair is made from nutrients in the body, and nutritional deficiencies can cause hair damage and hair loss. For example, biotin deficiencies have been linked to hair loss and skin disorders, and sufficient levels of the B vitamins are necessary for hair health and growth.

Many Western diets are lacking in nutrients. The whey protein concentrate formulation utilized in this study contains structural nutrition and proteins to help make hair stronger, longer, healthier and with less breakage. It also contains nutrients and herbs specifically selected for their positive effects on hair, including these nutrients per 32 g serving:
Niacin (vitamin B3) 200% DV (Daily Value) improves circulation including blood flow in the scalp
Folate 100% DV a B vitamins to help reduce the impact of stressors
Vitamin B 12 100% DV a B vitamins to help reduce the impact of stressors
Biotin 833% DV Deficiency can cause hair loss (REF); abundance can help hair growth
Zinc 133% DV Enhances immune function, which can stimulate hair growth
Manganese 100% DV essential to proper hair growth; deficiency is associated with hair loss
Foti Root (Ho Shou Wu or Polygonum multiflorum) a popular Chinese herb traditionally used for many reasons, including to darken pre mature gray hair, hair darkening, hair thickening and hair regrowth.
Kudzo Root Chinese herb, the second richest plant source of isoflavenones which researchers believe may have a positive effect on blood flow and microcirculation
Pumpkin Seed may help regulate testosterone levels
Honeysuckle Flower Helpful for skin health
Chrysanthemum Flower Used in Chinese medicine—may have anti inflammatory properties
PABA may help restore graying hair to its original color

EWPC contains the following amino acids: L Methionine, L Cysteine, Silica, L Cystine, L Isoleucine, L Arginine, L Leucine, L Lysine, L Phenylalanine, L Threonine, L Valine, L Aspartic acid, L Serine, L Glutamic Acid, L Proline, L Glycine, L Alanine, L Tyrosine, L Histidine. Amino acids are the building blocks of the body, including hair.

The subjects in this study were instructed to take EWPC immediately following their hair transplant each day for at least 3 months. Of the 115 subjects in the study group, 85 had their first hair transplant, and 30 had their second (“second sitting”) or subsequent hair transplant.

This study aims to demonstrate that taking EWPC reduced the telogen phase after a hair transplant, therefore achieving results more quickly using an all natural approach.

Materials and Methods

EWPC as per the present invention was used in this experiment. EWPC contains whey protein concentrate, and was free from whey protein isolates and growth hormones. The supplement contains additional vitamins, herbs and amino acids.
EWPC Nutrition facts. Serving Size: 1 Scoop (32 g).

| | Amount Per | | |
|---|---|---|---|
| | Serving (1 | | |
| | Scoop) | | % Daily Value |
| Calories | 150 | | |
| Calories from Fat | 8 | | |
| Total Fat | 1 | g | 1.35% |
| Cholesterol | 5 | mg | 1.83% |
| Total Carbohydrates | 11 | g | 3.66% |
| Protein | 14 | g | 26.40% |
| Niacin (as nicinamide) | 40 | mg | 200% |
| Folate (as folic acid) | 400 | mcg | 100% |
| Vitamin B-12 (as cyanocobalamin) | 6 | mcg | 100% |
| Biotin | 2.5 | mg | 833% |
| Pantothenic acid (as D-calcium | 9 | mg | 90% |
| pantothenate) | | | |
| Iodine (as potassiumiodide) | 100 | mcg | 67% |
| Zinc (as zinc oxide) | 20 | mg | 133% |
| Manganese (as manganesecitrate) | 2 | mg | 100% |
| PABA | 30 | mg | ** |
| Proprietary Blend | 255 | mg | ** |

** Daily Value Not Established * Percent daily values based on a 2,000 calorie diet. L Methionine, L Cysteine, Foti Root (Ho Shou Wu), Kudzo Root, Pumpkin Seed, Honeysuckle Flower, Chrysanthemum Flower, Silica, L Cystine, L Isoleucine, L Arginine, L Leucine, L Lysine, L Phenylalanine, L Threonine, L Valine, L Aspartic acid, L Serine, L Glutamic Acid, L Proline, L Glycine, L Alanine, L Tyrosine, L Histidine

| | |
|---|---|
| Calcium -- 28% | Iodine -- 33.5% |
| Potassium -- 3% | Sodium -- 2% |
| Phosphorus -- 24% | Magnesium -- 4% |
| Chlorophyll (none in vanilla flavor) -- ** | Chloride -- 1% |

** daily value not established

Other Ingredients: Whey protein concentrate, natural flavors, stevia and xylitol

Flavors: chocolate, vanilla

EWPC is free of many common allergens: no wheat, no gluten, no soy, no eggs, no nuts, no peanut oil, and no tree nuts. (Whey is a milk product.)

EWPC has no artificial sweeteners, no xanthan gum, no MSG, no artificial colors, and no artificial flavors. It is sweetened with all natural stevia and xylitol.

The subjects were adults aged 20 to 72. The study group was composed of 21% women and 79% men. They were selected at random from a group of patients seeking hair transplants. The subjects were not compensated, and were given a supply of EWPC. They were all healthy with no serious underlying medical conditions. A full medical history of each subject was taken. They did not have any underlying medical conditions at the time of the hair transplant that would adversely affect their hair. The subjects did not have thyroid problems, iron deficiency and/or anemia.

Additionally, these subjects were also not taking any lifestyle products that might adversely affect their hair, such as Whey Protein Isolate (WPI), using testosterone patches, or ingesting or injecting growth hormones, fat burners, fad diets, soy products, creatine, or anabolics. All subjects were screened for allergies to Iodine, PABA, taking sulfonamides, pregnant, lactating or have liver disease, drink excessive alcohol, smoke, uncontrolled diabetics, lactose intolerance. All were told not to use any other hair vitamins especially those with biotin because we do not want them to exceed 5 mg day.

All subjects were given our normal preoperative sheet of avoiding aspirin, alcohol, spicy foods, and vitamins so as to avoid bleeding and to do a 2 week vigorous massage on the top and back of their head with a Conair massager for maximum elasticity and blood flow. All subjects were treated with slit incisions with blades at various sizes. At no time was a 16 gauge needle or punch utilized.

The subjects took EWPC daily for a minimum of 3 months. They were instructed to ingest 2 scoops (64 g) of EWPC in water or a beverage each day.

Six weeks after their hair transplant, each subject was contacted and asked if they had any hair growth in the transplanted area (the transplanted hair follicles). These same subjects were also questioned about the length of their transplanted hair 3 months (approximately 12 weeks) after their hair transplant.

Results

All 115 of the subjects (100%) in the study group who complied with the instructions had 1-2 hairs starting to grow in the transplanted area 6 weeks after their hair transplant. The control group that did not take EWPC did not have hair starting to grow from the transplanted hair at 6 weeks.

Three months (approximately 12 weeks) after their hair transplant, all the subjects (100%) had significant growth of at least ¾″ length of hair in the transplanted area of 1-2 hairs. The transplanted hair of the control group had little or no significant growth.

Note: During interviews, many patients commented that they were pleased to see hair growth so quickly after their hair transplants, and that they did not have a long waiting period before getting results.

Special Case

Of the original 124 study subjects, 9 dropped out of the study because they did not comply with taking EWPC, due to lactose intolerance or not wanting to follow the protocol. One of these subjects took half the recommended amount of EWPC mixed with Whey Protein Isolate (Whey Protein Isolate may be linked to hair loss and hair damage).

TABLE
Post-Hair Transplant Growth of Transplanted Hair

| | Growth starting of 1- | |
|---|---|---|
| | 2 hairs appearing 6 | Significant |
| | weeks post- | growth of 1-2 hairs at |
| | transplant Some | least ¾″ 12 weeks post- |
| | growth 6 weeks post- | transplant At least ¾″ |
| Group | transplant | 12 weeks post-transplant |
| Took formulation | 100% | 100% |
| n = 115 | | |
| Control - no | 0% | little or no significant |
| formulation | | growth 0% |
| n = 115 | | |

The growth hormone free whey protein concentrate formulation was shown to significantly reduce the length the dormancy (telogen) period after a hair transplant from more than 12 weeks to 6 weeks. Faster results after a hair transplant have not been proven by any other therapy in the hair transplant field.

Clearly, nutritional supplementation can have a strong positive impact on the speed of results after a hair transplant. EWPC contains additional nutrients that may be associated with improved hair, including biotin, B vitamins, zinc, and an amino acid blend. EWPC contains the Chinese herbal remedies Kudzo Root and Foti Root (Ho Shou Wu) which are traditionally used for many ailments including hair problems. The formula also includes pumpkin seed, honeysuckle flower and chrysanthemum flower.

In addition to the natural form of whey and the extra ingredients, EWPC is a completely natural product, with no artificial sweeteners, no xanthan gum, no MSG, no artificial colors, and no artificial flavors. It contains the natural sweeteners stevia and xylitol.

The test composition had a significant perceived benefit as compared to the whey protein isolate on hair and nails for nearly all study participants.

Various tests and preliminary results have also indicated efficacy in treating hair loss from alopecia areata, chemotherapy treatment, and poor nutrition, especially in elderly patients.

In one embodiment, whey protein in the composition and method of the present invention is replaced with egg whites, casein, or a combination thereof.

In another embodiment, the composition and method of the present invention utilizes whey protein as described herein, egg whites, and casein in a therapeutically beneficial proportion.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. A composition comprising:
whey protein concentrate 60-75% w/w;
biotin 0.1-2% w/w;
potassium iodide, 0.0001-0.1% w/w;
folic acid, 0.001-0.1% w/w;
paraaminobenzoic acid (PABA), 0.05-0.5% w/w;
L-methionine, 0.0001-0.1% w/w;
L-cysteine, 0.05-0.3% w/w;
said composition providing greater therapeutic hair growth and reduced adverse side effects than compositions comprising whey protein isolates.

2. The composition of claim 1 further comprising flavoring agents.

3. The composition of claim 1 further comprising potassium iodide, niacinamide, Vitamin B12, Vitamin B5, Zinc Oxide, Manganese Citrate, Foti, Kudzu Root, Pumpkin Seed, Honeysuckle, Chrysanthemum flower and silica.

4. A method of promoting and increasing hair growth in humans comprising:
providing an initial dose of a hair growth composition to be ingested, wherein said composition is administered in a 32 g dose and contains:
whey protein concentrate 20-25 g;
biotin 2 - 10 μg;
Folic Acid 2-10 μg;
PABA 25-45 mg;
L-Methionine 0.5-2.0 mg;
L-Cysteine 25-45 mg;
administering daily subsequent 32 g doses for a period of 2 weeks-6 months; wherein said method increases hair growth.

5. A composition comprising:
casein, egg whites, or combinations thereof 60-75% w/w;
biotin 0.1-2% w/w;
potassium iodide, 0.0001-0.1% w/w;
folic acid, 0.001-0.1% w/w;
paraaminobenzoic acid (PABA), 0.05-0.5% w/w;
L-methionine, 0.0001-0.1% w/w;
L-cysteine, 0.05-0.3% w/w;
said composition providing greater therapeutic hair growth and reduced adverse side effects than compositions comprising whey protein isolates.
